(19) United States
(12) Patent Application Publication
(10) Pub. No.: US 2007/0077312 A1
(43) Pub. Date: Apr. 5, 2007

(54) Silver- and zinc- containing body care agent

(75) Inventors: Thorsten Berchert, Forchheim (DE); Michael Wagener, Bremen (DE); Peter Steinruecke, Erlangen (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Bio-Gate AG, Nuremberg (DE)

(21) Appl. No.: 10/569,709

(22) PCT Filed: Aug. 26, 2004
(86) PCT No.: PCT/EP04/09534

§ 371 Date: Oct. 2, 2006

(30) Foreign Application Priority Data
Aug. 29, 2003 (DE) 10340276.4

(51) Int. Cl. A61K 33/38; A61K 33/34; A61K 33/32

(52) U.S. Cl. 424/618; 424/641; 424/630

(57) ABSTRACT

The invention relates to a body care agent containing metal particles which release zinc and silver ions in said body care agent by contacting body liquid or body humidity and whose metallic silver content at least equal or greater than 99% (m/m).

The invention relates to a body care composition, a process for the production of such a body care composition, and a use for the production of a medicament for treating an inflammation and/or infection.

JP 11060417 A discloses inorganic oxide powders which can be used in cosmetics. The powder particles have a size of under 1 μm and are coated on their surfaces with a number of oxides of, for example, zinc or silver. The particles can be coated with a silver/zinc alloy of 20 to 80% w/w (percent by weight) of silver and 80 to 20% w/w of zinc, the weight of the coating being 0.1 to 10% w/w. After coating, the particles are heated at 300 to 400° C. in air for approximately one hour. It is to be assumed that owing to this treatment the silver-zinc coating is at least partially present in the form of oxides. The silver ions contained in the particles are present to a large part in the form of silver oxide. The release of silver ions by these particles depends very much on the chemical and biological environment. For example, the silver oxide can be converted to silver sulfide in an appropriate biological environment. Silver ions can then no longer be released.

DE 39 32 469 C2 and JP 04170960 A disclose hydroxyapatite which contains adsorbed silver and zinc ions. According to JP 04170960 A, the proportion of zinc is at least 5% w/w compared to the silver. The main disadvantage of the use of hydroxyapatite as a carrier for silver and zinc ions is that hydroxyapatite acts as an ion exchanger. This leads to the ions bound thereto being released depending on the ion concentration in the environment. The release of the ions is therefore difficult to control and not constant in a body care composition which is exposed to changing ion concentrations in the environment.

WO 00/06208 A1 discloses a toothpaste which contains antimicrobial ceramic particles or zeolite, wherein some of the exchangeable ions are replaced by antimicrobially active silver and zinc ions. These particles also act as ion exchangers and have the abovementioned disadvantages.

DE 101 41 117 A1 and U.S. Pat. No. 6,143,318 A1 disclose glasses as carriers for zinc and silver ions. These glasses release the zinc and silver ions in the manner of an ion exchanger. They therefore also have the abovementioned disadvantages.

WO 02/17984 A1 discloses an antimicrobial material for implanting into bone or for coating or producing an implant or an implantable medical device. In this case, particles formed from an antimicrobial metal are finely dispersed in a matrix material forming a matrix in the hardened state. The metal can be formed from one or more of the following constituents: Ag, Au, Pt, Pd, Ir, Sn, Cu, Sb, Zn.

WO 00/78281 A1 discloses an antimicrobial body care composition which contains an organic matrix in a part contacting human or animal skin and/or mucosa. This matrix contains homogeneously dispersed particles of metallic silver. The particles in this case have a size of between 1 and 50 nm and are contained in an amount which provides an antimicrobially active but less than cytotoxic concentration on the surface of the part contacting the skin and/or mucosa. The body care composition can be, for example, an ointment or a cream.

The object of the present invention is to make available an alternative body care composition having improved activity. In particular, the body care composition should have a relatively constant antimicrobial and optionally also antiinflammatory action over a long period of time.

This object is achieved by the features of patent claims 1, 22 and 25. Expedient embodiments result from the features of patent claims 2 to 21, 23, 24 and 26 to 46.

According to the invention, a body care composition is provided which contains metallic particles from which zinc ions and silver ions are released in the body care composition or on contact with body fluid or body moistness. The zinc ions and silver ions do not in this case necessarily have to be released in each case from one and the same particle. The ions mentioned already have an antimicrobial action at the lowest concentrations. Metallic particles, i.e. particles which consist of metal, can release a small amount of zinc ions and silver ions in a comparatively constant manner over a long period of time. By this means, formation of a possibly harmful concentration of zinc or silver ions in the skin and/or mucosa can be avoided. The particles have a content of metallic silver of at least 99% w/w (percent by weight). With such a high silver content, a cytotoxic effect of other metal ions, in particular of copper ions, does not come to bear. The percentage details indicating the metal content relate here and below, if not stated otherwise, to the proportion by weight of the metals indicated in the total weight of the particles. They are details of percentages by weight (% w/w).

To achieve the antimicrobial and optionally also antiinflammatory action, it has proven advantageous if the content of metallic silver is at least 99% w/w and accordingly the content of metallic zinc is at most 1% w/w. An antiinflammatory action can be achieved if the particles are contained in a higher concentration in the body care composition than is necessary for achieving a merely antimicrobial action. The particles are a depot for zinc and silver ions, which can release these ions over a long period of time under customary use conditions of a body care composition. Moreover, metallic particles allow the amount of silver ions made available from the particles according to JP 11060417 A to be made available with a significantly lower particle size. Owing to the smaller size of the particles, these are more stable to sedimentation and can be blended better into body care compositions, such as, for example, oils or ointments. Since body care compositions often have to be stable in their composition over long periods of time, this is a significant advantage of the particles employed in the body care composition according to the invention.

Body care compositions are products which are brought into contact with human or animal skin and/or mucosa in order to achieve cleansing, protective, therapeutic, curative, caring, cosmetic or alleviating action. Examples are the products which customarily have surfaces which contact the skin and consist of a natural or synthetic polymer material. These can be, for example, absorbent disposables, such as feminine hygiene articles, in particular sanitary napkins, panty liners or tampons, incontinence liners, diapers, training panties, medical bandages, plasters, nonwovens, textiles, celluloses, toothbrushes or pacifiers. The body care compositions can be produced from a natural substance, such as wool, viscose, cellulose and derivatives derived therefrom or natural rubber or can contain these natural substances. They can also be produced from plastics or contain plastics which contain the particles. The plastics can be, for example: polyethylenes and copolymers derived therefrom, polypropylenes and polyblends produced therefrom, polybutenes, polystyrenes in homo- and copolymers, acrylic/butadiene/styrene terpolymer (ABS), synthetic rubber, hard and soft PVC, polytetrafluoroethene (PTFE), polychlorotrifluoro-ethylene (PCTFE) and other fluoropolymers, polyvinyl ethers, polyvinyl acetates, polyvinyl propionates, polyvinyl alcohols, copolymers of vinyl alcohol, polyvinylacetals, polyethylene glycols, acrylic polymers, methyl polymethacrylate, polyacrylonitrile, polycyanoacrylates, polymers based on polymeth-acrylimide, polyacrylimides, polyvinylamines, poly-amides including polyphenylene isophthalamide, poly(p-phenylene terephthalamide), linear polyurethanes and polyesters including polyethylene terephthalate (PET), polybutylene terephthalate (PBT) and polytetramethylene terephthalate (PTMT), polycarbonates and polymers derived therefrom, polyoxymethylenes (POM), polyethers, polyether ether ketones, polyether block amides, condensation resins, such as phenolic plastics and aminoplastics, crosslinked polyesters including polyester resins, epoxy resins, crosslinked polyurethanes, reaction resins based on methyl methacrylate, polysiloxanes and other polymers having an inorganic main chain.

The body care compositions can also be preparations, in particular medicinally active preparations, such as emulsions, lotions, gels, creams, ointments, healing ointments, powders, cosmetics, skin protection creams or ointments, disinfectants or antiinflammatory medicaments, suspensions, soaps, synthetic surfactants, bath additives, peeling preparations, face lotions, dental care compositions, toothpastes, mouthwashes, tooth-cleaning chewing gums, prosthesis adhesives, hair shampoos, sunscreen compositions, etc. These products often contain a polymer or an organic constituent in a carrier, which can be a good substrate for a multiplicity of microorganisms. Growth of these microorganisms in these substrates can cause hygienic or medical problems.

The particles can be contained in the body care composition in an amount which makes possible an antimicrobially active but less than cytotoxic concentration of zinc ions and silver ions in a site of contact of the body care composition.

The particles can be present in the body care composition such that zinc or silver ions are released only on contact with body moistness of the skin and/or mucosa. This can, for example, be the case if the particles are present in an oily or fatty preparation in which they have no contact with water or if they are contained in a dry body care composition, such as a plaster. Body fluid or body moistness can be, for example, skin moistness, blood, perspiration, lymph, menstrual fluid or urine. The ions in the body care composition can also be released independently of contact with body fluid or body moistness. This can, for example, be the case if the body care composition is a gel or a cream and the particles release the ions in an aqueous phase contained therein.

The body care composition according to the invention has an antimicrobial and optionally also antiinflammatory action. Moreover, because of the antimicrobial action of the metal ions, it needs no preservatives in addition to the particles. It can be designed as an, in particular medicinal, healing or care ointment, cream or gel. Because of the antiinflammatory action, such a preparation can be used medicinally alternatively to corticoid-containing preparations. As a hand ointment, cream or gel, the antimicrobial action also protects against the transmission of germs, e.g. by shaking hands, and prevents the invasion of germs in the case of small wounds on the hands. By virtue of the fact that preservatives can be dispensed with, fewer, in particular allergic, intolerability reactions moreover occur. By virtue of the fact that in addition to the antimicrobially active silver ions antimicrobially active zinc ions are also released from the particles, a particularly effective body care composition is made available. The zinc ions and the silver ions mutually assist each other in the antimicrobial action. This lies, inter alia, in the fact that they have a different specificity for microorganisms in their antimicrobial action. Moreover, zinc in combination with silver has a particularly good wound-healing and antiinflammatory action. The reason for this could be that the growth of the microorganisms disrupting the wound-healing, whose growth is not inhibited by zinc ions alone, is suppressed by the silver. The body care composition according to the invention is suitable in particular for patients who must permanently take increased care over body care and body hygiene. These can, for example, be persons who have a weakened immune system and/or an increased risk of suffering from skin infections, such as, for example, diabetics.

It is advantageous for the body care if more silver ions than zinc ions are released from the particles in a defined time unit. The defined time unit can, for example, be the time during contact of the body care composition with the skin and/or mucosa or contact with the body fluid or body moistness. The defined time unit can, for example, be one, 4, 8 or 12 hours. Preferably, copper ions can also be released from the particles in the body care composition or on contact with body fluid or body moistness. Copper ions also have an antimicrobial action, the spectrum of action differing from that of the zinc ions and silver ions. By this means, an even better wound-healing and antiinflammatory action can be achieved than with the combination of silver and zinc ions. Preferably, more silver ions than copper ions are released from the particles during the time unit. Furthermore, it is advantageous if more zinc ions than copper ions are released during the time unit. In order to release more ions of a first metal (e.g. silver) than ions of a second metal (e.g. zinc), the particles can, for example, contain a larger amount of the first metal than of the second metal.

The particles preferably have a content of metallic silver of at least 99.5% w/w. As a result of such a silver content, the body care composition becomes even more tolerable, because side effects of other metal ions, in particular a cytotoxic effect, e.g. of copper ions, or the induction of allergies, almost do not take place.

The particles can contain up to 0.5% w/w of metallic zinc. Metallic copper can also be present up to a content of 0.5% w/w. Preferably, the particles are formed of a silver-zinc alloy or of a silver-zinc-copper alloy. Preferably, the particles contain impurities of less than 5 ppm of potassium, sodium or chlorine. Greater impurities in the silver can cause undesired side-effects.

The particles contained in the body care composition preferably have a diameter of 1 to 50 nm, in particular 5 to 15 nm, preferably 10 nm. This makes it possible to make available a body care composition, such as an ointment or a cream, which contains no excipients for dispersing the particles. Furthermore, it has been discovered that the metal ions have a preserving action. Therefore, in addition to the particles, preservatives can also be dispensed with. Undesirable, in particular allergic, reactions induced by a dispersing excipient or a customary preservative, such as, for example, formaldehyde, can thereby be avoided.

The particles can at least partly also be porous particles containing metallic silver having a mean diameter of between 1 and 100 μm. The mean internal porosity of the particles can be at least 65%, in particular between 65 and 95%, preferably between 65 and 90%, in particular between 70 and 85%, preferably between 75 and 85%, or preferably between 85 and 95%, in particular between 90 and 95%. Owing to their size of 1 to 100 μm, on normal use of the body care composition the particles are barely or not at all able to penetrate from outside, e.g. through the skin, into the bloodstream of a human or mammal. The antimicrobial effect is restricted solely to the skin surface. The induction of allergies and undesired toxic effects is thereby avoided. At the same time, the porosity of the particles guarantees that an antimicrobially and optionally also anti-inflammatorily active concentration of silver, zinc and optionally copper ions can be made available by the particles. These ions especially act on the surface of the skin or mucous membrane contacting the body care composition and have no negative influence on underlying tissue. The particles are by this means and because of their size, which prevents penetration into the skin, very skin-tolerable and biocompatible. The body care composition is thereby suitable in particular for patients who must permanently take increased care over body care and body hygiene, such as, for example, diabetics.

Internal porosity is understood as meaning the percentage proportion of the volume of the particle which is not filled by metal. The mean internal porosity of the particles can be determined by the following process:
1. Embedding of the particles in a plastic,
2. Preparation of ultrathin sections of the embedded particles,
3. Taking of transmission electron microscope (TEM) photographs of the particles,
4. Determination of the percentage proportion of the area not filled by metal in each case within a particle in relation to the total area of this particle in a majority of the TEM photographs and
5. Calculation of the mean value of a majority of percentage proportions determined in this way.

The step lit. 4 can in this case be carried out by means of a computer-assisted image analysis of the TEM photographs. In addition to the internal porosity, the total porosity of the particles can also be determined. For this purpose, the tap density of a powder of the particles is first determined. The tap density is the mass of one volume unit of a powder bedded as tightly as possible by tapping. The tap density can be determined according to DIN ISO 3953. The value determined in the course of this is calculated as the percentage proportion of the density of the metal forming the particles, here silver having a density of 10.49 g/cm³, and subtracted from 100%. The value calculated in this way represents the total porosity of the particles. For the particles contained in the body care composition according to the invention, it can lie between 85 and 95%, in particular between 90 and 95%, preferably between 93 and 95%.

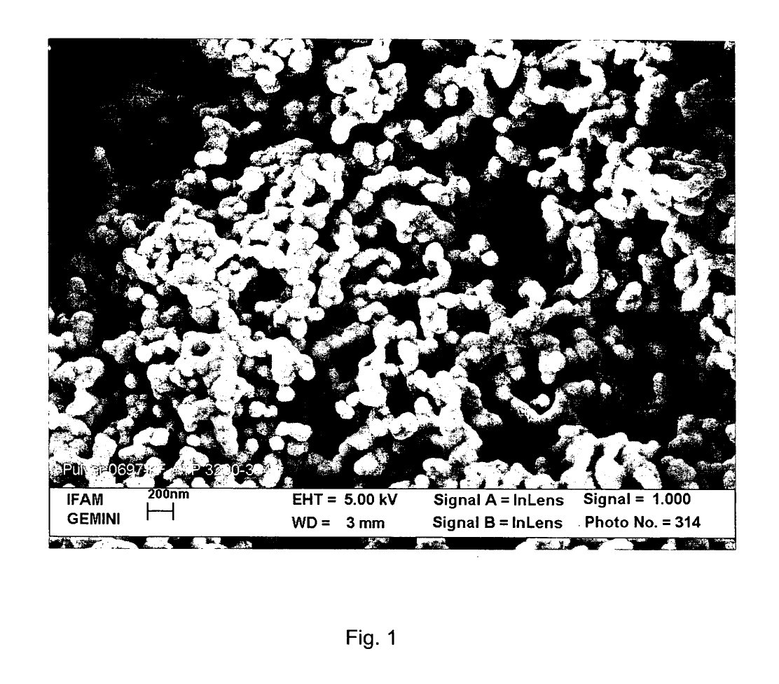

The porous particles are particularly well-suited for a long-lasting comparatively constant release of the silver and zinc ions if they are present as agglomerates of metallic primary particles. The agglomerates can be produced by thermal evaporation of the metal forming the agglomerates and subsequent deposition of the metal vapor on a metal filter. Preferably, the agglomerates are formed from primary particles having a mean diameter of between 10 and 200 nm, preferably between 15 and 80 nm. Primary particles of this size allow an adequate release of silver ions and can be easily prepared. The primary particles can be identified by electron microscopy on the basis of their external shape and size. They are to be seen, for example, as spheroidal structures in FIG. 1. The primary particles are connected to one another by means of sinter necks. The mean distance between the in each case outermost primary particles on the surface of the agglomerates preferably lies in the range from 20 to 200 nm, preferably 100 to 200 nm.

The porous particles preferably have a spongy structure. As a result of the large surface area thereby made available, silver, zinc and optionally copper ions can be released in adequate amount in order to be antimicrobially and optionally also antiinflammatorily active.

Preferably, the particles have a mean outer diameter of 2 to 20 μm, preferably 2 to 5 μm. The specific surface area of the particles can be between 2 and 10 m²/g, in particular between 3 and 6 m²/g, preferably between 3.5 and 4.5 m²/g. The specific surface area can be determined volumetrically according to the BET method, for example by means of N₂ adsorption. The BET method is a method known according to Brunauer, Emmett and Teller for the determination of the surface area and optionally also of the pore size distribution of solid bodies (e.g. powders), which assumes that gases, vapors etc. are first adsorbed in a monomolecular layer on solid bodies with release of a measurable heat of adsorption. For example, the volume of nitrogen gas which is adsorbed at −196° C. on the adsorbent agent as a function of the pressure applied can be measured.

The particles can be contained in a carrier material which consists of a silicone oil, a mineral oil, glycerol or a customary ointment constituent known from pharmacology. For the preparation of a body care composition according to the invention, the particles can be prepared by a sputter process in which the metal forming the particles is evaporated and the metallic vapor is deposited directly into a carrier liquid, which is then incorporated into the body care composition.. The exceptional feature of the process consists in the fact that the evaporated metal is deposited not onto a solid carrier, such as, for example, powder particles, but into a liquid, which can be incorporated directly into the body care composition. The structure of the silver-zinc particles resulting during the course of this and the particle sizes achievable by means of this process are particularly advantageous for the incorporation of these particles into body care compositions. The carrier liquid can be a silicone oil, a mineral oil, glycerol or a customary ointment constituent known from pharmacology. The conglomerates can also be taken up in a carrier liquid of the type which is incorporated into the body care composition. Advantageously, the carrier liquid has a vapor pressure at 20° C. of less than 250 mbar, in particular less than 70 mbar, preferably less than 10 mbar, in particular less than 3 mbar.

Moreover, the invention relates to the use of metallic particles for production of a medicament for the treatment of an inflammation and/or infection in a mammal or human, the particles releasing zinc ions and silver ions in the medicament or on contact with body fluid or body moistness. The particles have a content of metallic silver of at least 99% w/w. Customary medicaments for the treatment of inflammation in a mammal or human often contain a combination of antiinflammatory and antimicrobial active compounds. The antimicrobial active compound should prevent or control an infection, in particular with Staphylococcus aureus. Customarily, the antimicrobial active compound is an antibiotic. Alternatively, the antibiotic can also be administered systemically, whereas the antiinflammatory active compound is administered locally, e.g. topically. Because of the danger existing, in particular on long-term use, of the formation of antibiotic resistance, the use of antibiotics, however, should be reduced to a minimum. As an antiinflammatory active compound, hitherto, for example, a corticoid such as cortisone was used which, however, has a large number of side-effects. The essential advantage of the medicament prepared according to the invention consists in the fact that the particles have both an antiinflammatory action and an antimicrobial action. The use of antibiotics can be reduced and the side-effects of the corticoids or other antiinflammatory active compounds can be avoided.

Treatment is preferably carried out topically, i.e. for example by application to the skin or a wound. The medicament can be an ointment, a cream or a gel. Further advantageous embodiments of the invention result from the foregoing details concerning the body care composition according to the invention.

The invention is illustrated below with the aid of exemplary embodiments:

FIG. 1 shows a scanning electron microscope view of a silver agglomerate and

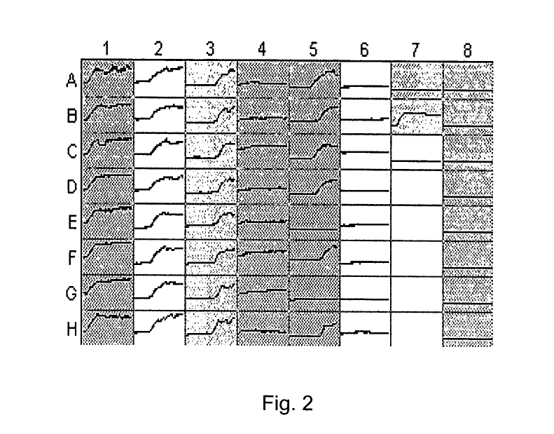

FIG. 2 shows a matrix of graphic representations of the time course of the growth of bacteria, measured in the form of optical density (OD) of a medium, in contact with various creamy body care compositions.

FIG. 1 shows a scanning electron microscope view of a silver agglomerate. The silver agglomerate here consists essentially of spherical primary particles having a mean particle size of approximately 60 nm. The primary particles are essentially connected to one another by means of sinter necks. They form a highly porous structure. The silver agglomerate shown here has a size of approximately 10 μm.

The results shown in FIG. 2 have been determined according to the process disclosed in DE 197 51 581 A1. This process is described further in Bechert, Thorsten et al., Nature Medicine (2000), Vol. 6, No. 8, pages 1053 to 1056. The disclosure content of the two aforementioned documents is included here. The body care compositions according to the invention to be tested were prepared in the form of creams, in each case applied to a material as a carrier and employed in the test as described. In detail, the test was carried out as follows:

First, various cream samples are prepared. To each carrier is applied an amount of 11 mg of the respective cream. Subsequently, 200 μl of a Staphylococcus epidermidis-containing solution are filled into each hollow of a microtiter plate. The carriers with the cream samples are in each case incubated in one of the hollows at 37° C. for one hour. The carriers are then removed and washed three times with physiological buffer. Subsequently, the carriers are in each case placed in a hollow of a microtiter plate which is filled with 200 μl of a minimal medium. The carriers are incubated at 37° C. for 24 hours. Subsequently, the carriers are removed and discarded. To each hollow of the microtiter plate are added 50 μl of a complete medium (Trypcase soya, BioMerieux, No. 69280, Marcy l' Etoile, France). Subsequently, the turbidity of the solution is measured at intervals of 30 minutes over a period of 48 hours. In the course of this procedure the solution is maintained at a temperature of 37° C. The turbidity measurement is carried out using light of a wavelength of 578 nm by means of a suitable reading apparatus. Turbidity indicates that bacteria have been released into the surroundings from the surface of the carrier.

For the preparation of the cream samples, the base cream used was “Cremaba Plus HT” from Spinnrad®, Certus Handels GmbH, 22848 Norderstedt, Germany. This is an emulsion base containing the following ingredients: Aqua, Caprylic/Capric Triglyceride, Pentylene Glycol, Hydrogenated Lecithin, Butyrospermum Parkii, Glycerin, Squalane, Ceramide 3. The following further constituent has been incorporated into the base cream:
silicone oil having a silver content of 0.65% w/w; the silver is present therein in the form of particles having a mean diameter of 10 nm; the silver is designated below as “nanodisperse silver”;
or
agglomerates of metallic silver present in powder form having a mean porosity of 80% and a mean diameter of 5 μm; the silver is designated below as “agglomerate silver”.

Creams containing 0.01% w/w of nanodisperse silver and containing 0.1% w/w and 0.5% w/w of agglomerate silver were prepared. Moreover, a cream containing 0.05% w/w of nanodisperse silver was prepared, where the nanodisperse silver here consisted of an alloy consisting of 99.5% w/w of silver, 0.49% w/w of zinc and 0.01% w/w of copper. Furthermore, a cream containing 1.5% w/w of agglomerate silver was prepared, where the agglomerate silver here consisted of an alloy consisting of 99.5% w/w of silver, 0.49% w/w of zinc and 0.01% w/w of copper.

For the preparation of the cream, the substances were in each case blended in a 50 ml beaker, heated in a water bath at 75° C. for 20 minutes and then dispersed for 5 minutes by means of an Ultraturrax (Janke and Kunkel, drive T25, stator diameter 25 mm, rotor diameter 17 mm). Subsequently, the cream was cooled and thoroughly mixed again.

In FIG. 2, each field shows an x-y graph in which the time is plotted on the x-axis and the optical density on the y-axis. The experimental results shown in columns 1 to 8 of FIG. 2 have been determined with the following creams in parallel experimental batches A to H corresponding to the rows A to H:
Column 1, rows A-H: cream without additions of silver
Column 2, rows A-H: cream with 0.1% w/w of agglomerate silver
Column 3, rows A-H: cream with 0.5% w/w of agglomerate silver
Column 4, rows A-H: cream with 1.5% w/w of agglomerate silver, consisting of 99.5% w/w of silver, 0.49% w/w of zinc and 0.01% w/w of copper
Column 5, rows A-H: cream with 0.1% w/w of nanodisperse silver
Column 6, rows A-H: cream with 0.05% w/w of nanodisperse silver, consisting of 99.5% w/w of silver, 0.49% w/w of zinc and 0.01% w/w of copper
Column 7, row A: positive control
Column 7, row B: negative control
Column 7, row C: blank
Column 8, rows A-H: sterile controls

A polymer containing metallic silver was employed in the positive control. The values show that the bacteria employed are sensitive to silver and can be killed thereby. In the negative control the same polymer was employed that, however, contained no silver. The blank is a value measured in an empty hollow of the microtiter plate, which was subtracted in the evaluation of all measurement values. In the sterile controls, in each case only medium without addition of Staphylococcus epidermidis was employed in order to show that the bacterial growth does not come from the medium.

The results can be summarized as follows:

| | Onset OD | Onset | |
|---|---|---|---|
| | [h] | OD [h] | |
| Sample description | gross | net | Action |
| 1A-H Cream without | 5.2 | 0 | not |
| silver additions | | | antibacterial |
| 2A-H Cream with 0.1% | 18.4 | 13.2 | highly |
| w/w of agglomerate | | | antibacterial |
| silver | | | |
| 3A-H Cream with 0.5% | 32.2 | 27.0 | highly |
| w/w of agglomerate | | | antibacterial |
| silver | | | |
| 4A-H Cream with 1.5% | 37.9 | 32.7 | highly |
| w/w of agglomerate | | | antibacterial |
| silver, consisting | | | |
| of 99.5% w/w of | | | |
| silver, 0.49% w/w of | | | |
| zinc and 0.01% w/w | | | |
| of copper | | | |
| 5A-H Cream with | 35.3 | 30.1 | highly |
| 0.01% w/w of | | | antibacterial |
| nanodisperse silver | | | |
| 6A-H Cream with | Limit | >42.8 | bactericidal |
| 0.05% w/w of | | | |
| nanodisperse silver, | | | |
| consisting of 99.5% | | | |
| w/w of silver, 0.49% | | | |
| w/w of zinc and | | | |
| 0.01% w/w of copper | | | |
| 7A/B Positive | Limit/9.2 | — | OK |
| control /negative | | | |
| control | | | |
| 8A-G Sterile | Limit | — | OK |
| controls | | | |
| 7C Blank | | — | OK |

“Onset OD [h] gross” designates the time measured in hours until an exponential increase in the optical density (OD) of around 0.2 occurred. “Onset OD [h] net” results from “Onset OD [h] gross” by respective subtraction of the value “Onset OD [h] gross” determined for the cream without additions of silver. In parallel experimental batches, the mean value is indicated in each case. “Antibacterial” designates an action in which the growth of the bacteria is retarded, while “bactericidal” designates an action in which the bacteria are killed to 100%, such that bacterial growth can no longer be observed.

The experimental results show that agglomerate silver, like nanodisperse silver, has a highly antibacterial action. Nanodisperse silver, at relatively low silver concentrations, is as active as agglomerate silver. With agglomerate silver, however, a highly antibacterial action can still be achieved. Both the action of the agglomerate silver and the action of the nanodisperse silver is increased in the creams which, alongside silver, additionally contain zinc and copper.

1-45. (canceled)

46. A body care composition comprising metallic particles, wherein the metallic particles comprise metallic zinc and metallic silver, wherein the total weight of metallic particles is at least 99 percent metallic silver, wherein the metallic particles release zinc ions and silver ions, and wherein the release of ions takes places in the body care composition, on contact with body fluid or body moistness, or any combination thereof.

47. The body care composition as claimed in claim 46, wherein more silver ions than zinc ions are released from the metallic particles in a defined time unit.

48. The body care composition of claim 46, wherein the metallic particles further comprise metallic copper, and wherein the metallic particles further release copper ions.

49. The body care composition of claim 48, wherein more silver ions than copper ions are released from the metallic particles in a defined time unit.

50. The body care composition of claim 46, wherein the total weight of metallic particles is at least 99.5 percent metallic silver.

51. The body care composition of claim 48, wherein the total weight of metallic particles is up to 0.5 percent metallic zinc and up to 0.5 percent metallic copper.

52. The body care composition of claim 46, wherein the metallic particles comprise at least one of a silver-zinc alloy and a silver-zinc-copper alloy.

53. The body care composition of claim 46, wherein the metallic particles comprise impurities of less than 5 ppm of potassium, sodium or chlorine.

54. The body care composition of claim 46, wherein the diameter of the metallic particles is 1 to 50 nm.

55. The body care composition of claim 46, wherein the diameter of the metallic particles is 5 to 15 nm.

56. The body care composition of claim 46, wherein the diameter of the metallic particles is 10 nm.

57. The body care composition of claim 46, wherein the body care composition comprises no excipients for dispersing the metallic particles.

58. The body care composition of claim 46, wherein the body care composition comprises no preservatives in addition to the metallic particles.

59. The body care composition of claim 46, wherein the metallic particles comprise porous particles comprising metallic silver, and wherein the mean diameter of the porous particles is between 1 and 100 μm.

60. The body care composition of claim 59, wherein the mean inner porosity of the porous particles is at least 65%.

61. The body care composition of claim 59, wherein the mean inner porosity of the porous particles is between 65% and 95%.

62. The body care composition of claim 59, wherein the mean inner porosity of the porous particles is between 65% and 90%.

63. The body care composition of claim 59, wherein the mean inner porosity of the porous particles is between 70% and 85%.

64. The body care composition of claim 59, wherein the mean inner porosity of the porous particles is between 75% and 85%.

65. The body care composition of claim 59, wherein the mean inner porosity of the porous particles between 85% and 95%.

66. The body care composition of claim 59, wherein the mean inner porosity of the porous particles is between 90% and 95%.

67. The body care composition of claim 59, wherein the porous particles comprise agglomerates of metallic primary particles.

68. The body care composition of claim 67, wherein the mean diameter of the primary particles is between 10 nm and 200 nm.

69. The body care composition of claim 67, wherein the mean diameter of the primary particles is between 15 nm and 80 nm.

70. The body care composition of claim 67, wherein the mean distance between the outermost primary particles on the surface of the agglomerates is between 20 nm to 200 nm.

71. The body care composition of claim 67, wherein the mean distance between the outermost primary particles on the surface of the agglomerates is between 100 nm to 200 nm.

72. The body care composition of claim 59, wherein the metallic particles have a spongy structure.

73. The body care composition of claim 59, wherein the mean outer diameter of the metallic particles is from about 2 μm to 20 μm.

74. The body care composition of claim 59, wherein the mean outer diameter of the metallic particles is from about 2 μm to 5 μm.

75. The body care composition of claim 59, wherein the metallic particles have a specific surface area of between 2 m2/g and 10 m2/g.

76. The body care composition of claim 59, wherein the metallic particles have a specific surface area of between 3 m2/g and 6 m2/g.

77. The body care composition of claim 59, wherein the metallic particles have a specific surface area of between 3.5 m2/g and 4.5 m2/g.

78. The body care composition of claim 46, wherein the composition further comprises a carrier material and wherein at least one component of the carrier material is selected from the group consisting of silicone oils, mineral oils, glycerols, an ointment constituent, and any combination thereof, and wherein the metallic particles are contained within the carrier material.

79. The body care composition of claim 46, wherein the body care composition is selected from the group consisting of an emulsion, a lotion, a gel, a cream, an ointment, a healing ointment, a powder, a cosmetic, a skin protection cream or ointment, a disinfectant, a suspension, a soap, a synthetic surfactant, a bath additive, a peeling preparation, a face lotion, a dental care composition, a toothpaste, a mouthwash, a tooth-cleaning chewing gum, a prosthesis adhesive, a hair shampoo, a sunscreen composition, and any combination thereof.

80. The body care composition of claim 79, wherein the composition is operatively associated with a substrate selected from the group consisting of an absorbent disposable article, a feminine hygiene article, an incontinence liner, a diaper, training panties, a medical bandage, a plaster, a nonwoven, a fabric, a cellulose, a toothbrush, a pacifier, and any combination thereof.

81. The body care composition of claim 80, wherein the feminine hygiene article is selected from the group consisting of a sanitary napkin, a panty liner, a tampon, and any combination thereof.

82. A sputtering process for the production of a body care composition, the composition comprising:
evaporating zinc and silver metal to form a metallic vapor;
depositing the metallic vapor directly in a carrier liquid; and
forming a body care composition with the carrier liquid into which the metallic vapor has been deposited; the resulting body care composition comprising metallic particles, wherein the metallic particles comprise metallic zinc and metallic silver, wherein the total weight of metallic particles is at least 99 percent metallic silver, wherein the metallic particles release zinc ions and silver ions, and wherein the release of ions takes places in the body care composition, on contact with body fluid or body moistness, or any combination thereof.

83. The process of claim 82, the carrier liquid comprises at least one component selected from the group consisting of silicone oils, mineral oils, glycerols, an ointment constituent or any combination thereof.

84. The process of claim 82, wherein the carrier liquid at 20° C. has a vapor pressure of less than 250 mbar.

85. The process of claim 82, wherein the carrier liquid at 20° C. has a vapor pressure of less than 70 mbar.

86. The process of claim 82, wherein the carrier liquid at 20° C. has a vapor pressure of less than 10 mbar.

87. The process of claim 82, wherein the carrier liquid at 20° C. has a vapor pressure of less than 3 mbar.

88. A method of treating at least one of inflammation and infection in a mammal, the method comprising:
topically applying to a mammal an effective amount of body care composition comprising metallic particles, wherein the metallic particles comprise metallic zinc and metallic silver, wherein the total weight of metallic particles is at least 99 percent metallic silver, wherein the metallic particles release zinc ions and silver ions, and wherein the release of ions takes places in the body care composition, on contact with body fluid or body moistness, or any combination thereof, and wherein the topical application of the composition results in at least one of a decrease of inflammation and a decrease of infection.

89. The method of claim 88, wherein the body care composition is an ointment, a cream, a gel, or any combination thereof.

90. The method of claim 88, wherein the mammal is a human.
